(19) United States
(12) Patent Application Publication
(10) Pub. No.: US 2022/0157453 A1
(43) Pub. Date: May 19, 2022

(54) MEDICAL POWER SUPPLY SYSTEM, INFORMATION PROCESSING APPARATUS, INFORMATION PROCESSING METHOD, IMAGE PROCESSING APPARATUS, AND IMAGE PROCESSING METHOD

(71) Applicant: SONY GROUP CORPORATION, TOKYO (JP)

(72) Inventors: YUKI SUGIE, TOKYO (JP); MASAYA TAKEMOTO, TOKYO (JP); MASAHITO YAMANE, TOKYO (JP)

(21) Appl. No.: 17/439,613

(22) PCT Filed: Mar. 13, 2020
(86) PCT No.: PCT/JP2020/011007

§ 371 Date: Sep. 15, 2021

(30) Foreign Application Priority Data
Mar. 27, 2019 (JP) 2019-060222

(51) Int. Cl. G16H 40/67; H04N 5/232; G06F 3/14; G06V 20/00; H04N 7/18; A61B 17/00; A61B 90/00

(52) U.S. Cl. CPC G16H 40/67; H04N 5/232411; G06F 3/14; H04N 2005/2255; H04N 7/18; A61B 17/00; A61B 90/361; G06V 20/00

(57) ABSTRACT

The present technology relates to a medical power supply system, an information processing apparatus, an information processing method, an image processing apparatus, and an image processing method capable of controlling power consumption.

A medical power supply system including a first information processing apparatus that is connected to a medical device installed in an operation room and a second processing apparatus that exchanges data with the first information processing apparatus, further includes: an acquisition unit that acquires data from the medical device; a determination unit that determines a use state of the medical device on the basis of the data; and a control unit that controls a power supply state of at least one medical device in the operation room on the basis of the use state of the medical device determined by the determination unit. The present technology can be applied to an information processing apparatus that controls power consumption of a device connected to an uninterruptible power supply.

### TECHNICAL FIELD

The present technology relates to a medical power supply system, an information processing apparatus, an information processing method, an image processing apparatus, and an image processing method, and for example, relates to a medical power supply system, an information processing apparatus, an information processing method, an image processing apparatus, and an image processing method configured to appropriately control power of a medical device.

### BACKGROUND ART

In many cases, power consumed by a medical device is normally supplied from a commercial AC power supply, and is supplied from an uninterruptible power supply (UPS) when an abnormality occurs in the commercial AC power supply, for example, when an instantaneous voltage drop (instantaneous interruption), a sudden power outage, or the like occurs.

In a case where the commercial AC power supply is normal, the UPS can supply power from the commercial AC power supply to a load device such as a medical device, and in a case where the commercial AC power supply is abnormal, the UPS can switch its output and supply power from a built-in battery to the load device. As a result, even if an abnormality occurs in the commercial AC power supply, the load device to which the UPS is connected can use power supplied from the battery in the UPS. Therefore, the load device can continue the processing as it is.

Patent Document 1 discloses that in an X-ray imaging system, power distribution is dynamically controlled according to an operating status of a device.

### CITATION LIST

### Patent Document

Patent Document 1: Japanese Patent Application Laid-Open No. 2005-57997

### SUMMARY OF THE INVENTION

### Problems to be Solved by the Invention

Since an UPS has limited power supply capacity, it is desirable to save used capacity (connected capacity) as much as possible so that lack of capacity will not occur and power supply will not be interrupted when power is supplied from the UPS to a medical device. In addition, it is desired to perform appropriate power control for saving used capacity.

The present technology has been made in view of such a situation, and is intended to enable appropriate control in power supply.

### Solution to Problems

A medical power supply system according to one aspect of the present technology is a medical power supply system including: a first information processing apparatus that is connected to a medical device installed in an operation room; and a second processing apparatus that exchanges data with the first information processing apparatus, the medical power supply system further including: an acquisition unit that acquires data from the medical device; a determination unit that determines a use state of the medical device on the basis of the data; and a control unit that controls a power supply state of at least one medical device in the operation room on the basis of the use state of the medical device determined by the determination unit.

An information processing apparatus according to one aspect of the present technology includes: an acquisition unit that acquires data from a medical device; a determination unit that analyzes the data and determines a use state of the medical device; and a control unit that controls a power supply state of the medical device on the basis of the use state of the medical device determined by the determination unit.

An information processing method performed by an information processing apparatus that controls a power supply state of a medical device according to one aspect of the present technology includes: acquiring data from the medical device; analyzing the data and determining a use state of the medical device; and controlling the power supply state of the medical device on the basis of the use state of the medical device that has been determined.

An image processing apparatus according to one aspect of the present technology includes: an acquisition unit that acquires video data captured by a medical device; a determination unit that analyzes the video data and determines a use state of the medical device; and a control unit that controls a power supply state of the medical device on the basis of the use state of the medical device determined by the determination unit.

An image processing method performed by an image processing apparatus that is connected to a medical device and processes video data from the medical device according to one aspect of the present technology includes: acquiring video data captured by the medical device; analyzing the video data and determining a use state of the medical device; and controlling a power supply state of the medical device on the basis of the use state of the medical device that has been determined.

The medical power supply system according to one aspect of the present technology includes: the first information processing apparatus connected to a medical device installed in an operation room; and the second processing apparatus that exchanges data with the first information processing apparatus. Data is acquired from the medical device, a use state of the medical device is determined on the basis of the data, and a power supply state of at least one medical device in the operation room is controlled on the basis of the use state of the medical device.

In the information processing apparatus and the information processing method according to one aspect of the present technology, data is acquired from the medical device, the data that has been acquired is analyzed, the use state of the medical device is determined, and the power supply state of the medical device is controlled on the basis of the use state of the medical device that has been determined.

In the image processing apparatus and the image processing method according to one aspect of the present technology, video data captured by the medical device is acquired, the video data is analyzed, the use state of the medical device is determined, and the power supply state of the medical device is controlled on the basis of the use state of the medical device that has been determined.

Note that each of the information processing apparatus and the image processing apparatus may be an independent apparatus, or may be an internal block constituting one apparatus.

Furthermore, a program can be provided by transmitting the program through a transmission medium or by recording the program on a recording medium.

### BRIEF DESCRIPTION OF DRAWINGS

FIG. 1 is a diagram illustrating a configuration example of an embodiment of a medical power supply system to which the present technology is applied.

FIG. 2 is a diagram for explaining an IPC.

FIG. 3 is a diagram for explaining functions of the medical power supply system.

FIG. 4 is a diagram for explaining first operation of the medical power supply system.

FIG. 5 is a diagram for explaining a first process of a server.

FIG. 6 is a diagram for explaining a process based on a table for an endoscope system.

FIG. 7 is a diagram for explaining another process based on the table for the endoscope system.

FIG. 8 is a diagram for explaining a process based on a table for an ultrasound image diagnosis system.

FIG. 9 is a diagram for explaining a process based on a table for an operative field camera.

FIG. 10 is a diagram for explaining a process based on a table for an operating place camera.

FIG. 11 is a diagram for explaining a position of a monitor determined to be unused.

FIG. 12 is a diagram for explaining a process based on a power supply management table.

FIG. 13 is a diagram illustrating an example of the power supply management table.

FIG. 14 is a diagram for explaining a second process of the server.

FIG. 15 is a diagram for explaining a process based on a table for a plurality of devices.

FIG. 16 is a diagram for explaining functions of the medical power supply system.

FIG. 17 is a diagram for explaining second operation of the medical power supply system.

FIG. 18 is a diagram for explaining a third process of the server.

FIG. 19 is a diagram for explaining a fourth process of the server.

FIG. 20 is a diagram for explaining functions of the medical power supply system.

FIG. 21 is a diagram for explaining third operation of the medical power supply system.

FIG. 22 is a diagram for explaining a configuration example of a general-purpose personal computer.

### MODE FOR CARRYING OUT THE INVENTION

Hereinafter, embodiments for implementing the present technology (hereinafter, referred to as embodiments) will be described.

### <Configuration Example of Medical Power Supply System>

The present technology can be applied to an apparatus that controls supply of power and a system including such an apparatus. Furthermore, a case where power is supplied by an uninterruptible power supply (UPS) will be described as an example.

An example of the device that operates if power is supplied thereto is a device configured such that power is normally supplied from a commercial AC power supply and power is supplied from a UPS when an abnormality occurs in the commercial AC power supply, for example, an instantaneous voltage drop (instantaneous interruption) or a sudden power outage or the like occurs. In particular, there are many devices for medical use to which UPSs are connected because it is necessary to prevent a situation in which supply of power is stopped due to a power outage or the like and the devices do not operate.

Here, a case of enabling appropriate supply of power when a UPS is connected to a device for medical use and power is supplied from the UPS to the device will be described as an example. However, the following description is an example, and the present technology can also be applied to a case where supply of power to a device other than a medical device is controlled or power is supplied from, for example, a commercial AC power supply other than a UPS.

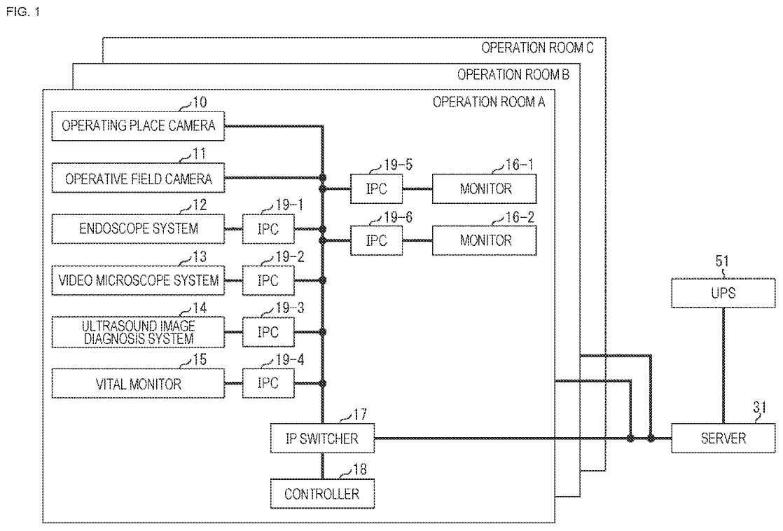

FIG. 1 is a diagram illustrating a configuration of an embodiment of a medical power supply system including an information processing apparatus and an image processing apparatus to which the present technology is applied.

In the following description, an expression a device for medical use (medical device) is used, and it is assumed that the medical device is a device installed in a hospital. For example, various devices described as being installed in an operation room A in FIG. 1 are medical devices.

The medical power supply system illustrated in FIG. 1 performs control related to power supply of medical devices installed in three operation rooms A to C. Medical devices are installed in each of the operation rooms A to C. Here, the medical devices installed in the operation room A will be described as examples.

In the operation room A, an operating place camera 10, an operative field camera 11, an endoscope system 12, a video microscope system 13, an ultrasound image diagnosis system 14, a vital monitor 15, a monitor 16-1, a monitor 16-2, an internet protocol (IP) switcher 17, and a controller 18 are installed. Furthermore, the endoscope system 12, the video microscope system 13, the ultrasound image diagnosis system 14, the vital monitor 15, the monitor 16-1, and the monitor 16-2 are connected to Internet Protocol Converters (IPCs) 19-1 to 19-6, respectively.

In the following description, in a case where it is not necessary to distinguish between the monitor 16-1 and the monitor 16-2, they are simply referred to as monitors 16. Furthermore, in a case where it is not necessary to distinguish the IPCs 19-1 to 19-6 from one another, they are simply referred to as IPCs 19.

The operating place camera 10, the operative field camera 11, the IPCs 19-1 to 19-6, the IP switcher 17, and the controller 18 are connected via a network, and are configured to be able to exchange data therebetween. The network is configured in a wired and/or wireless manner. The IP switcher 17 is connected to a server 31 and is configured to be able to exchange data with the server 31.

The server 31 is connected to a UPS 51 and monitors switching of a power supplying device to the UPS 51, capacity, and the like. The server 31 controls a power-on/off state of a device to which power is supplied from the UPS 51 to be described later, and processes an image from a device installed in the operation room A. The UPS 51 is connected to each device via a dedicated line or the like.

The UPS 51 is generally a power supply system that protects important systems in various scales ranging from large-scale systems and facilities, for example, an online system of a financial institution and an Internet data center, to computer devices and network devices such as a server and a personal computer, from a power outage and power supply troubles (failures).

The UPS 51 includes a combination of a power conversion unit (electric circuit) called a rectifier or an inverter and a power storage unit (mainly a battery). When a power outage or an instantaneous voltage drop occurs, the UPS keeps supplying stable power by using power stored in a storage battery, and the function as a power conversion prevents a power supply trouble from affecting a device. In particular, a medium-scale or higher UPS having a power stabilization capability is sometimes referred to as a constant voltage & constant frequency (CVCF) because it is one of the missions thereof to supply a “constant voltage/constant frequency” to a load apparatus.

Furthermore, as the types of the UPS 51, there are types called a continuous inverter power supply system UPS, a line-interactive UPS, a continuous commercial power supply system UPS, and the like. For example, the continuous inverter power supply system UPS is a system in which stable output that is always adjusted is output to a connected device by using an inverter regardless of the state of commercial power, and is called a continuous inverter power supply system because normally the inverter circuit always supplies power.

In the continuous inverter power supply system UPS, a power supply failure occurring in a commercial power supply does not affect a device, and even when a power outage occurs, clean power can be supplied without generating an interruption time at the time of operation switching. Furthermore, since continuous inverter operation is possible, it is also a feature thereof that the backup time can be extended by adding a battery.

As the UPS 51 constituting the medical power supply system, a continuous inverter power supply system UPS can be used. Furthermore, other systems such as the line-interactive UPS and the continuous commercial power supply system UPS can also be applied.

In general, among devices installed in a hospital, a device that needs to keep operating even when an emergency such as a power outage occurs is inserted into an uninterruptible outlet (outlet connected to a UPS), a device power supply to which may be instantaneously interrupted is inserted into an outlet of an emergency power supply, and the other devices are normally inserted into commercial outlets. Even in a case where the present technology is applied, it is possible to perform power distribution in a manner physically determined in advance as described above.

The operating place camera 10 images the inside of the operation room. The operative field camera 11 mainly images an operative site and images the inside of the operation room including the operative site. In a case where each of the operating place camera 10 and the operative field camera 11 is an IP camera, each of the operating place camera 10 and the operative field camera 11 is connected to the IP switcher 17 without the IPC 19 therebetween. FIG. 1 illustrates an example where it is assumed that each of the operating place camera 10 and the operative field camera 11 is an IP camera and is connected to the IP switcher 17 without an IPC 19 therebetween.

The endoscope system 12 is a device that is inserted into a body cavity from outside the body to observe and to photograph an inner surface of the body cavity and a surface of an organ. The types of the endoscope system 12 can be roughly classified into a rigid endoscope including a metal tube and a flexible fiberscope including a freely bendable optical fiber. The endoscope system 12 includes a rigid endoscope or a fiberscope and includes a monitor that displays an image obtained from the rigid endoscope or the fiberscope.

Examples of the endoscope system 12 includes a laryngoscope, a bronchoscope, an esophagoscope, a gastroscope, a gastrocamera, and a duodenoscope inserted from a mouth, a rectoscope inserted from an anus, a urethroscope and a cystoscope inserted from a urethra, a laparoscope, a thoracoscope, and a mediastinoscope inserted by adding a small incision to an abdominal wall or a chest wall, and the like, and the present technology can be applied to any of them.

The video microscope system 13 is a device for enlarging and observing an operative site in a surgical operation for a minute region in, for example, neurosurgery, ophthalmology, and the like. The video microscope system 13 includes an imaging element and includes an electronic imaging microscope unit capable of electronically imaging an operative site. Furthermore, the video microscope system 13 is configured such that a video of the operative site captured by the microscope unit is displayed on the monitor installed in the operation room, and an operator can perform a medical operation while observing the video of the operative site displayed on the monitor.

The ultrasound image diagnosis system 14 is a device that examines a state (form, kinetics, function) in a living body by using ultrasound, and is a system that obtains a cross-sectional image of a body by transmitting ultrasound to the body and visualizing reflection (echo) from tissue.

The vital monitor 15 is a device that continuously measures and monitors a vital sign of a patient. The vital monitor 15 measures and monitors, for example, a heart rate, a pulse, a blood pressure, a body temperature, an electrocardiogram, a blood oxygen concentration, and the like.

Videos from these devices such as a video obtained by the endoscope system 12 are displayed on a monitor included in the endoscope system 12 and the monitor 16. When the operator wants to check the video obtained by the endoscope system 12, the operator checks the monitor showing the video from the endoscope system 12. When the operator wants to check the vital monitor 15, the operator checks the vital monitor 15.

As described above, if it is necessary to check different monitors in order to check individual videos, there is a possibility that efficiency of a medical operation is lowered. Therefore, by simultaneously displaying a plurality of medical images on a monitor, it is possible to look over various pieces of information at once and to improve efficiency of a medical operation.

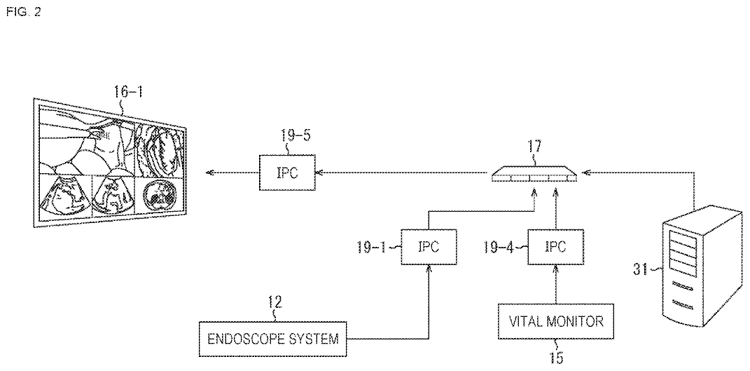

A system for simultaneously displaying a plurality of medical images on the monitor will be further described with reference to FIG. 2. FIG. 2 illustrates part of the medical power supply system illustrated in FIG. 1. Since the medical power supply system illustrated in FIG. 2 includes the IPC 19, it is possible to record and manage images in a hospital, synthesize recorded and managed images and images during a medical operation into one sheet of image, and present the combined image to an operator.

The endoscope system 12 outputs the captured stream image to the IPC 19-1 as an image signal such as YCC/422/10 bit of 3 Gbps-serial digital interface (3G-SDI), for example. Note that the format of the image signal is not limited to YCC/422/10 bit of the 3G-SDI, and may be various other formats. Similarly, the vital monitor 15 outputs the obtained signal to the IPC 19-1.

Each of the IPC 19-1 and the IPC 19-4 functions as the IPC 19 on the transmission side. Each of the IPC 19-1 and the IPC 19-4 encodes an image signal constituting a stream image that has been supplied into a stream format such as a low latency video codec (LLVC), for example, converts the encoded image signal into an IP packet, and outputs the IP packet as a network signal to the IPC 19-5 or the server 31 via the IP switcher 17.

The type of encoding of a stream image may be other than LLVC, and may be, for example, H264 (MPEG-4 Part 10 Advanced Video Coding), Joint Photographic Experts Group 2000 (JPEG 2000), Differential Pulse Code Modulation (DPCM), or the like.

The server 31 is, for example, an operation room (OR) server or the like provided in a hospital, and acquires via the IP switcher 17, stores and manages various types of images (so-called multi-modality images) such as a computed tomography (CT) image, a magnetic resonance imaging (MRI) image, and an X-ray image captured in advance in the hospital, and outputs the images to the IPC 19 via the IP switcher 17 as necessary. Furthermore, as will be described later, the server 31 also performs control to turn off a device or bring the device into a power saving state.

The IPC 19-5 functions as the IPC 19 on the receiving side. The IPC 19-5 processes images transmitted from the other IPCs 19 via the IP switcher 17 and various images supplied from the server 31 into one image, and outputs the image to the monitor 16-1 as an image signal so that the image is displayed on the monitor 16-1.

With such a configuration, the IPC 19 combines a CT image, an MRI image, an X image, and the like imaged in a hospital in advance, an operative field, an endoscopic image, a laparoscopic image, and the like into one sheet of image and displays for the operator the combined image on the monitor 16 to present necessary information to the operator. Here, LLVC and H264 are visually lossless compression methods, and JPEG 2000 and DPCM are lossless compression methods. Therefore, in the case of combining images into one PinP image, the encoding method may be JPEG 2000 or DPCM for an image combined as a main image, for example, an operative field image, an endoscopic image, a laparoscopic image, or the like, and LLVC or H264 may be used for an image to be a sub image, for example.

### <Function of Medical Power Supply System, First Operation>

Functions and operation of the medical power supply system illustrated in FIG. 1 will be described. In the following description, power supply management performed by the medical power supply system will be described. Furthermore, in power supply management, the video obtained from the IPC 19 is analyzed, and on the basis of the analysis result, the device installed in the operation room is turned off or is shifted to the power saving state.

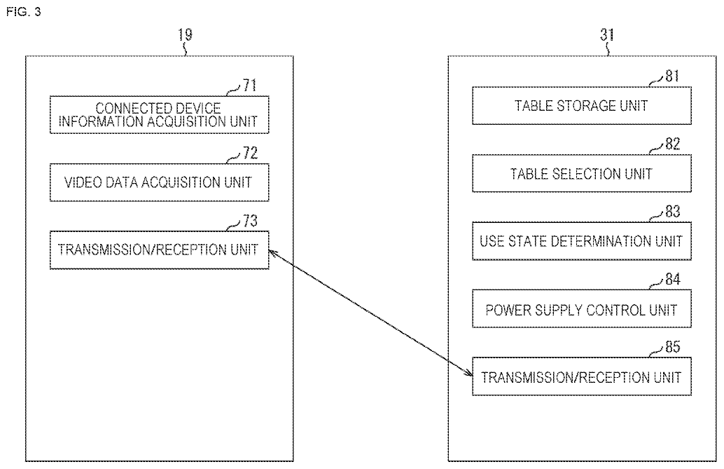

FIG. 3 illustrates a functional configuration example of the medical power supply system. The IPC 19 includes a connected device information acquisition unit 71, a video data acquisition unit 72, and a transmission/reception unit 73. The server 31 includes a table storage unit 81, a table selection unit 82, a use state determination unit 83, a power supply control unit 84, and a transmission/reception unit 85.

The connected device information acquisition unit 71 acquires information of a device to which the IPC 19 is connected (hereinafter referred to as connected device information). The connected device information acquisition unit 71 may have a function of storing connected device information that has been acquired. The video data acquisition unit 72 acquires video data of a video captured by the device to which the IPC 19 is connected. The transmission/reception unit 73 transmits connected device information and video data to the server 31, and receives data from the server 31.

The table storage unit 81 stores a determination table and a power supply management table to be described later. The table selection unit 82 selects, for example, a table associated with the device indicated by connected device information from a plurality of tables stored in the table storage unit 81.

The use state determination unit 83 analyzes video data transmitted from the IPC 19 side on the basis of the table selected by the table selection unit 82, and determines the use state of the device. The power supply control unit 84 performs control to turn off the device determined to be unused by the use state determination unit 83 or shift the device to the power saving state. The transmission/reception unit 85 exchanges data with the IPC 19.

As first operation of the medical power supply system illustrated in FIG. 3, a case where the server 31 analyzes the video from the IPC 19 and sets a device whose power supply is controlled will be described as an example.

The operation described below can be started when the power source to the medical device in the operation room is switched from the commercial AC power supply to the UPS 51. In other words, the operation described below can be executed when power distribution of the UPS 51 is appropriately performed in an emergency.

Furthermore, the operation described below can be executed not only in an emergency but also in a normal state, that is, when power is supplied from a commercial AC power supply to a medical device in an operation room.

That is, the present technology can also be applied to a case where power consumption can be appropriately limited at all times, and can also be applied to a case where power consumption can be appropriately limited in an emergency in which a power consumption amount needs to be suppressed particularly.

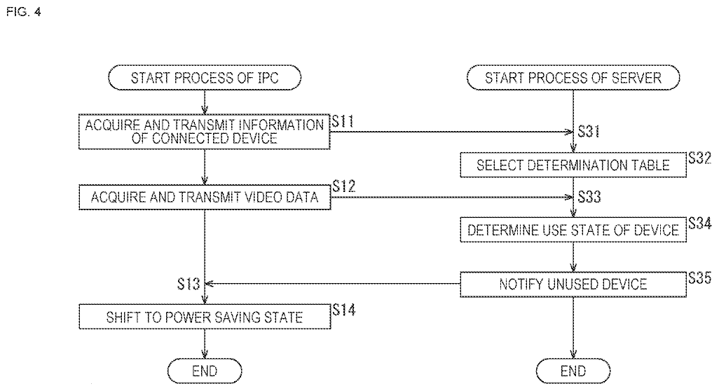

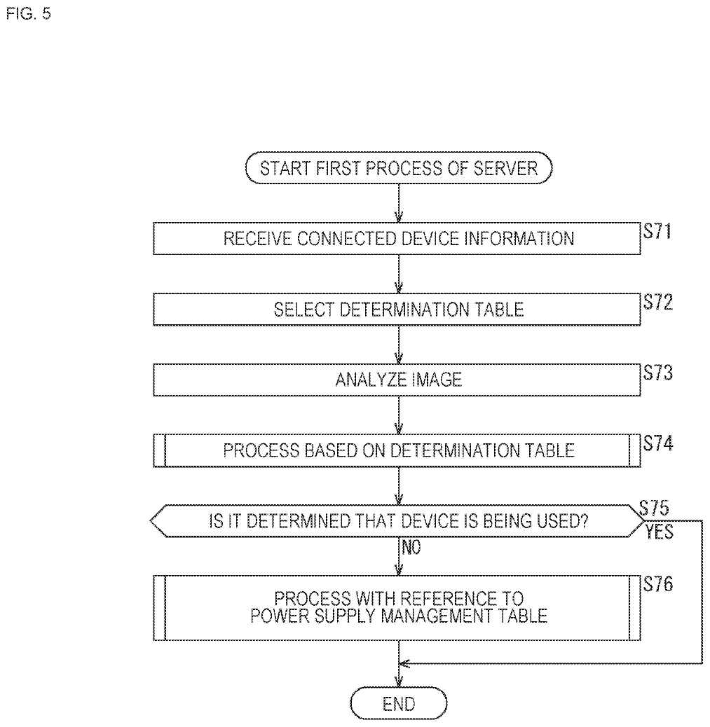

FIG. 4 is a diagram for explaining the first operation of the medical power supply system. The detailed operation of the server 31 will be described with reference to FIG. 5 and subsequent drawings.

In step S11, the connected device information acquisition unit 19 of the IPC 19 acquires information (connected device information) of the device to which the IPC 19 is connected, and causes the transmission/reception unit 73 to transmit the information to the server 31.

When the IPC 19 is connected to a device and is installed, for example, when the IPC 19 is connected to the endoscope system 12, connected device information indicating that the IPC 19 is connected to the endoscope system 12 is set. The connected device information that has been set may be acquired in step S11.

Furthermore, the medical device generates image data and data associated with the image data (hereinafter referred to as metadata) in a format defined by Digital Imaging and COmmunications in Medicine (DICOM). DICOM is a standard specification that defines formats of medical images captured by the endoscope system 12, the video microscope system 13, and the like, and a communication protocol between medical image devices that handle the medical images.

Since metadata contains connected device information, the connected device information can be read by referring to the metadata. The IPC 19 can be configured such that in a case where the IPC 19 acquires image data and metadata associated with the image data from the connected device, the IPC 19 acquires connected device information by reading the connected device information contained in the metadata that has been acquired.

The IPC 19 transmits the connected device information that has been acquired to the server 31. If the transmission/reception unit 85 receives the connected device information from the IPC 19 in step S31, the server 31 advances the processing to step S32.

In step S32, the table selection unit 82 of the server 31 selects the determination table corresponding to the device indicated by the connected device information from among the plurality of determination tables stored in the table storage unit 81.

The table storage unit 81 of the server 31 has a determination table for each device. For example, the table storage unit 81 has a determination table for the operative field camera 11, a determination table for the endoscope system 12, and the like. The determination table is a table for determining whether or not the device is being used.

It is assumed that an expression, a device is being used, means that the device is used for its original purpose, and does not include a state in which the device is simply turned on. Furthermore, the expression, when a device is used for its original purpose, means when the device is used for a medical operation such as imaging the inside of a body cavity in the case of the endoscope system 12, for example.

In step S12, the video data acquisition unit 72 of the IPC 19 acquires video data from the connected device, and causes the transmission/reception unit 73 to transmit the video data to the server 31. If the transmission/reception unit 85 receives the video data from the IPC 19 in step S33, the server 31 advances the processing to step S34.

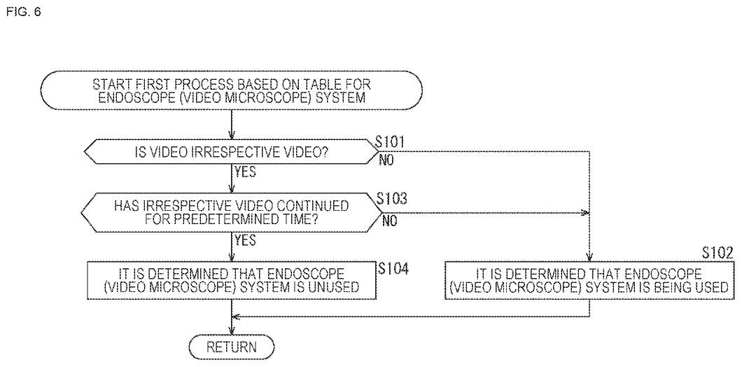

In step S34, the use state determination unit 83 of the server 31 determines the use state of the device. The use state determination unit 83 of the server 31 analyzes the video data transmitted via the IPC 19 on the basis of the determination table selected in step S32, and determines the use state of the device. The manner of this determination will be described later with reference to FIGS. 6 to 10.

In a case where it is determined in step S34 that there is a device not being used (unused), the processing proceeds to step S35. In step S35, the power supply control unit 84 of the server 31 notifies the unused device.

In step S13, if the IPC receives notification from the server 31, the IPC 19 advances the processing to step S14. In step S14, the IPC 19 instructs the connected device to transition to the power saving state.

Here, transition to the power saving state is taken as an example; however, for example, a message prompting power-off may be displayed on the monitor, or the device may be turned off.

### <First Process of Server 31>

The process of the server 31 will be further described with reference to FIG. 5.

In step S71, the transmission/reception unit 85 of the server 31 receives connected device information from the IPC 19. If the server 31 receives the connected device information from the IPC 19, the processing proceeds to step S72, and the determination table corresponding to the device indicated by the connected device information is selected by the table selection unit 82.

In step S73, the use state determination unit 83 performs image analysis by using video data from the connected device transmitted via the IPC 19 that has transmitted the connected device information. This image analysis is an analysis for extracting information required for performing the process based on the determination table executed in step S74.

In step S74, the use state determination unit 83 executes the process based on the determination table. The process based on the determination table executed by the use state determination unit 83 in step S74 will be described with reference to FIGS. 6 to 10.

Here, the process based on the determination table will be described with a flowchart. However, a lookup table may be prepared, and a determination process may be performed by referring to the lookup table.

### <Determination of Use State of Endoscope System, Microscope System>

The flowchart illustrated in FIG. 6 is a flowchart for explaining a process performed when the determination table for the endoscope system 12 is selected as the determination table. Note that, since the determination table for the endoscope system 12 can also be used as a determination table for the video microscope system 13, the description will be continued here assuming that a common determination table is used for the endoscope system 12 and the video microscope system 13.

Furthermore, in the following description, a case where connected device information indicates the endoscope system 12 and the determination table for the endoscope system 12 is selected will be described as an example; however, the present invention can also be applied to the video microscope system 13 by appropriately replacing the endoscope system 12 with the video microscope system 13.

In step S101, it is determined whether or not the video is an irrelevant video. The video captured by the endoscope system 12 is a video obtained when the inside of a body cavity such as an organ is imaged. Similarly, the video captured by the video microscope system 13 is also a video obtained when the inside of a body cavity such as an organ is imaged.

The server 31 analyzes the video data transmitted from the endoscope system 12 to determine whether or not the video is obtained when the inside of a body cavity such as an organ is imaged. For example, since the proportion of red (blood color) in the entire screen tends to be larger in videos obtained when the inside of a body cavity such as an organ is imaged, it may be determined whether or not a video is a video obtained when the inside of a body cavity such as an organ is imaged by determining whether or not the image contains a large red area.

Alternatively, it may be determined whether or not the video is a video obtained when the inside of a body cavity such as an organ is imaged. As described above, in a state where the endoscope system 12 is used, since the inside of a body cavity such as an organ is usually imaged, a video obtained when the inside of a body cavity such as an organ is imaged is obtained. Therefore, it is also considered that a video other than a video obtained when the inside of a body cavity such as an organ is imaged is obtained because the inside of a body cavity such as an organ is not imaged in a state where the endoscope system 12 is not used.

In a case where the endoscope system 12 is not used, the rigid endoscope or the fiberscope of the endoscope system 12 images a scene in an operation room such as a ceiling, a fluorescent lamp, a wall, a floor, an operator, or a staff member in the operation room. Here, a video such as a scene of the operation room other than the inside of a body cavity as described above is referred to as an irrelevant video.

Also in the video microscope system 13, similarly to the endoscope system 12, it is considered that the scene of the operation room other than the inside of the body cavity such as the floor is imaged when the video microscope system 13 is not used.

In step S101, it is determined whether or not the video is an irrelevant video. In a case where it is determined in step S101 that the video is not an irrelevant video, in other words, the video is a video obtained when the device is being used such as the video of the inside of a body cavity, the processing proceeds to step S102. In step S102, a determination result indicating that the device is being used is output.

In contrast, in a case where it is determined in step S101 that the video is an irrelevant video, in other words, the video is a video obtained when the device is unused such as the video of the scene of the operation room other than the inside of a body cavity, the processing proceeds to step S103.

In step S103, it is determined whether or not the irrelevant video continues for a predetermined time. In a case where it is determined in step S103 that the irrelevant video has not continued for the predetermined time, the processing proceeds to step S102, and a determination result indicating that the device is being used is output.

In contrast, in a case where it is determined in step S103 that the irrelevant video continues for the predetermined time, the processing proceeds to step S104, and a determination result indicating that the device is unused is output.

In a case where a determination result indicating that the device is unused is output, the process of shifting the device determined to be unused to the power saving state or a power-off state is executed in a subsequent process. Suppose the process of step S103 is omitted, that is, in a case where it is determined in step S101 that the video is an irrelevant video, the process proceeds to step S104. In a case where the determination result indicating that the device is unused is output, the device, in this case, the endoscope system 12 is shifted to the power saving state or the power-off state at the time point of capturing the irrelevant video.

Therefore, there is a possibility that an irrelevant video is imaged for example, in a state where the endoscope system 12 is not yet inserted into a body cavity at the time point when the endoscope system 12 is started to be used, or in a state where the endoscope system 12 is once taken out from the body cavity for some reason, and the endoscope system 12 shifts to the power saving state or the power-off state.

The determination process in step S103 is provided so that such a situation does not occur. By providing the determination process in step S103, it is possible to prevent the endoscope system 12 from shifting to the power saving state or the power-off state in a situation where it is not desirable that the endoscope system 12 shift to the power saving state or the power-off state.

In step S103, it is determined that the irrelevant video continues for a predetermined time when the device is unused. The predetermined time is set to a time sufficient for determining that there is no error in the determination result indicating that the device is unused. The measurement of the predetermined time may be performed by, for example, measuring time with a timer from a time point when the video is determined to be an irrelevant video, or may be performed by counting the number of frames and determining whether or not the number of frames has reached a predetermined number.

As described above, a use state of the endoscope system 12 (video microscope system 13) is determined.

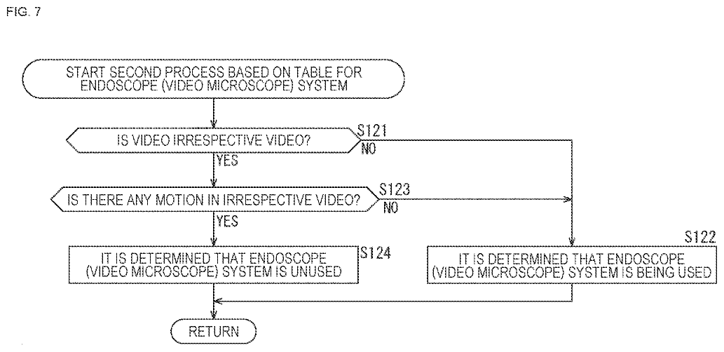

FIG. 7 is a flowchart for explaining the process in another determination table for the endoscope system 12 (video microscope system 13). The process based on the flowchart illustrated in FIG. 7 is different from the process based on the flowchart illustrated in FIG. 6 in the process of step S123 (FIG. 7) corresponding to step S103 (FIG. 6).

In step S121, as in step S101 (FIG. 6), it is determined whether or not the video is an irrelevant video. In a case where it is determined in step S121 that the video is not an irrelevant video, the processing proceeds to step S122, and a determination result indicating that the device is being used is output.

In contrast, in a case where it is determined in step S121 that the video is an irrelevant video, the processing proceeds to step S123. In step S123, it is determined whether or not there is no motion in the irrelevant video.

In a case where the endoscope system 12 is used, since the rigid endoscope or the fiberscope of the endoscope system 12 is in a state of being held by the operator, there is considerable camera shake or the like, and it is considered that there is motion in the video obtained from the endoscope system 12.

In other words, in a case where the endoscope system 12 is not used, it is considered that the rigid endoscope or the fiberscope of the endoscope system 12 is in a state of being placed at a predetermined location and is in a stationary state. Therefore, it is considered that there is no motion in the video obtained from the endoscope system 12 that is stationary.

Also in the video microscope system 13, similarly to the endoscope system 12, when the video microscope system 13 is not used, it is considered that there is no motion in the video obtained from the video microscope system 13.

For this reason, in step S123, it is determined whether or not there is no motion in the irrelevant video. For example, it may be determined whether or not there is no motion in the irrelevant video by analyzing a video from the endoscope system 12 and determining whether or not a motion vector is detected.

The determination as to whether or not there is no motion in the irrelevant video is made not for a momentary video but for a video in a predetermined time. In other words, it is determined whether or not a state in which there is no motion in an irrelevant video continues for a predetermined time.

In a case where it is determined in step S123 that there is motion in the irrelevant video, the processing proceeds to step S122, and a determination result indicating that the device is being used is output. In contrast, in a case where it is determined in step S123 that there is no motion in the irrelevant video, the processing proceeds to step S124, and a determination result indicating that the device is unused is output.

As described above, a use state of the endoscope system 12 (video microscope system 13) is determined.

Note that, here, the process based on the flowchart illustrated in FIG. 6 and the process based on the flowchart illustrated in FIG. 7 have been described as the processes of determining the use state of the endoscope system 12 (video microscope system 13). Only one of the two processes may be performed, or both the processes may be performed. Furthermore, in a case where both the two processes are performed, the processes may be performed in parallel, or one of the processes may be performed and then the other process may be performed.

### <Determination of Use State of Ultrasound Image Diagnosis System>

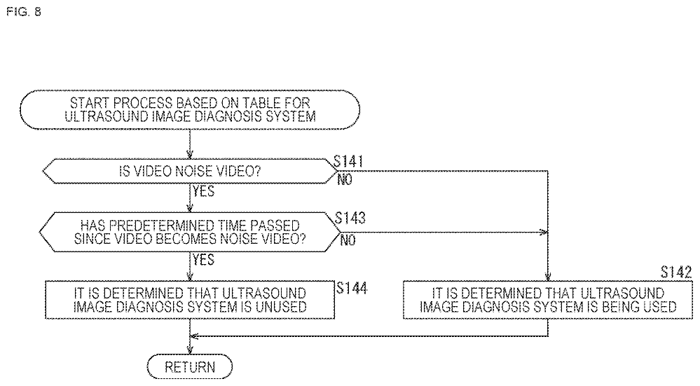

Next, determination of the use state of the ultrasound image diagnosis system 14 will be described with reference to FIG. 8. FIG. 8 is a flowchart for explaining the process performed when the determination table for an ultrasonic system is selected as the determination table.

In step S141, it is determined whether or not the video is a noise video. The video captured by the ultrasound image diagnosis system 14 is, for example, a video obtained by imaging an organ in the body while bringing an ultrasound generation unit (not illustrated) that generates an ultrasound wave into contact with the abdomen.

In contrast, when the ultrasound image diagnosis system 14 is not used, the ultrasound generation unit of the ultrasound image diagnosis system 14 is placed without being in contact with the abdomen or the like, and thus the obtained video becomes a noise video.

In step S141, it is determined whether or not the video is a noise video. In a case where it is determined in step S141 that the video is not a noise video, in other words, the video is a video obtained by imaging an organ or the like, the processing proceeds to step S142. In step S142, a determination result indicating that the ultrasound image diagnosis system 14 is being used is output.

In contrast, in a case where it is determined in step S141 that the video is a noise video, the processing proceeds to step S143.

In step S143, it is determined whether or not the noise video continues for a predetermined time. The reason why such a determination process is performed in step S143 is similar to the reason why the determination processes in step S103 (FIG. 6) and step S123 (FIG. 7) described above are performed, and is to prevent the determination that the device is unused even though the device is being used (in use).

In a case where it is determined in step S143 that the noise video has not continued for a predetermined time, the processing proceeds to step S142, and a determination result indicating that the device is being used is output. In contrast, in a case where it is determined in step S143 that the noise video has continued for the predetermined time, the processing proceeds to step S144, and a determination result indicating that the device is unused is output.

As described above, the use state of the ultrasound image diagnosis system 14 is determined.

### <Determination of Use State of Operative Field Camera>

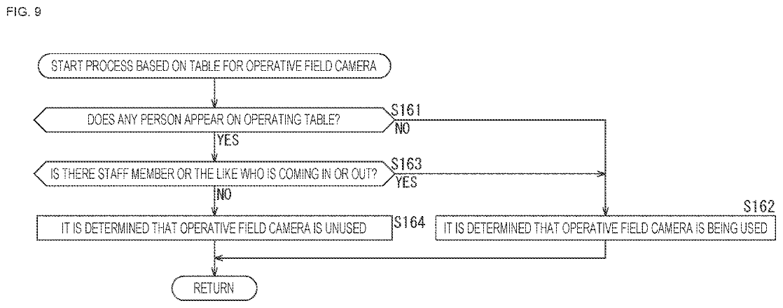

Next, determination of the use state of the operative field camera 11 will be described with reference to FIG. 9. FIG. 9 is a flowchart for explaining the process performed when the determination table for the operative field camera 11 is selected as the determination table.

In step S161, it is determined whether or not the video is a video in which a person is present on an operating table. Since the operative field camera 11 is a camera that images a field of view of a surgical site (operative field) in a medical operation such as thoracotomy or laparotomy, when the operative field camera 11 is being used, a patient lying on the operating table, and an operator, a staff member, or the like in the vicinity of the patient are imaged.

The state in which the operative field camera 11 is not used is considered to be a time when there is no patient or operator. In a case where the video captured by the operative field camera 11 is analyzed and the patient on the operating table is not imaged, in other words, in such a case where the operating table is imaged, it can be considered that the operative field camera 11 is not used.

Thus, in step S161, it is determined whether or not the video is a video in which a person is present on the operating table. In a case where it is determined in step S161 that a person appears on the operating table, the processing proceeds to step S162. In step S162, a determination result indicating that the operative field camera 1 is being used is output.

In contrast, in a case where it is determined in step S161 that the video is not a video in which a person is present on the operating table, the processing proceeds to step S163. In step S163, it is determined whether or not a staff member or the like is coming in or out. The video for this determination may be a video from the operative field camera 11 or a video from the operating place camera 10.

In a case where it is determined in step S163 that a staff member or the like is coming in or out, the processing proceeds to step S162, and a determination result indicating that the operative field camera 11 is being used is output. In contrast, in a case where it is determined in step S163 that no staff member or the like is coming in or out, the processing proceeds to step S164, and a determination result indicating that the operative field camera 11 is unused is output.

The reason why the determination process in step S163 is performed is similar to the reason why the process in step S103 (FIG. 6) described above or the like is performed, and is to prevent the determination that the operative field camera 11 is unused even though the operative field camera 11 is being used (in use).

In a case where the video from the operative field camera 11 is used, if a medical operation is being performed, since an operator or a staff member exists near the operating table, it is highly likely that the operator or the staff member appears in the video imaged by the operative field camera 11.

For example, if the process of step S161 is executed when a medical operation has not yet started and a patient is not yet on the operating table, it is determined that there is no person on the operating table. However, in this case, the operative field camera 11 is unused because a medical operation is being prepared, and it is considered that shifting to the power saving state is an erroneous determination.

In order to prevent such an erroneous determination, as described above, in step S163, it is determined whether or not a staff member or the like is coming in or not, and when it is determined that a staff member or the like is coming in or out, it is determined that the operative field camera 11 is being used even if there is no patient on the operating table.

Note that, as described above, the video used to execute the determination process in step S163 may be a video from the operating place camera 10. Since the operating place camera 10 images a wider range than the operative field camera 11 does, it is possible to determine whether or not a staff member or the like is coming in or out from an image obtained by imaging a wider range. Even if there is no person in the vicinity of the operating table, the present technology can also be applied to a case where a staff member or the like is coming in or out of the operation room for preparation or the like.

As described, it is also possible to determine the use state of a predetermined device, in this case, the operative field camera 11 by using the operating place camera 10 and the operative field camera 11. That is, a case where the use state of a predetermined device is determined by analyzing information from a plurality of devices is also within the scope of application of the present technology.

As described above, the use state of the operative field camera 11 is determined.

### <Determination of Use State of Device by Operating Place Camera>

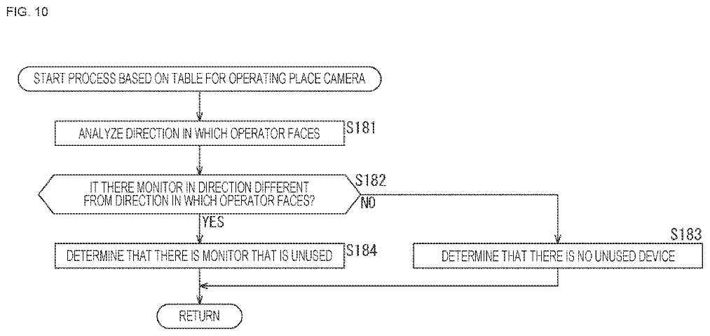

Next, determination of the use state of a device by the operating place camera 10 will be described with reference to FIG. 10. FIG. 10 is a flowchart for explaining a process performed when the determination table for the operating place camera 10 is selected as the determination table.

The operating place camera 10 is a camera that images the entire operation room. The description with reference to the flowcharts illustrated in FIGS. 6 to 9 are the processes for determining the use state of the endoscope system 12 itself and the use state of the ultrasound image diagnosis system 14 itself. The process described with reference to the flowchart of FIG. 10 is the process of detecting a device that is not being used in the operation room by analyzing a video captured by the operating place camera 10.

In step S181, the direction in which the operator faces are analyzed. For example, it is assumed that two monitors 16, the monitor 16-1 and the monitor 16-2 (FIG. 1), are installed in the operation room A.

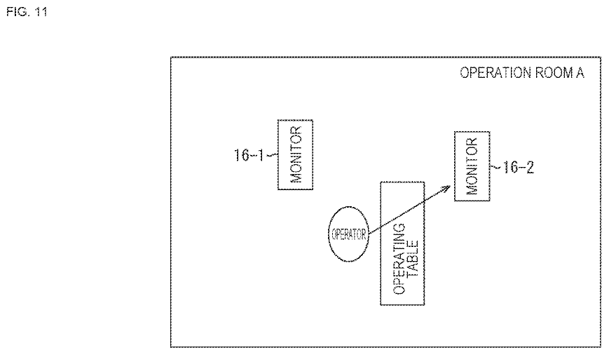

FIG. 11 illustrates an arrangement example of the monitors 16 in the operation room A. FIG. 11 is a view of the operation room A as viewed from the ceiling side. An operating table is arranged near the center of the operation room A, and the monitor 16-1 and the monitor 16-2 are arranged on the left side and the right side (in the drawing) of the operating table, respectively. In a case where the operator is located on the left side of the operating table and is performing a medical operation, the direction of the line-of-sight of the operator is toward the monitor 16-2 as indicated by an arrow in FIG. 11.

In such a case, the monitor 16-1 is located behind the operator. In the case of such a situation, since the monitor 16-1 located behind the operator is at a location that cannot be seen by the operator, it can be determined that the monitor 16-1 is unused.

In order to make such a determination, in step S181 (FIG. 10), the video captured by the operating place camera 10 is analyzed, and the direction in which the operator faces is detected. In step S182, it is determined whether or not the monitor 16 is located in a direction different from the direction in which the operator faces. As described with reference to FIG. 11, for example, in a case where the direction in which the operator faces is the rightward direction in FIG. 11, it is determined whether or not the monitor 16 is located in a direction different from the rightward direction in which the operator faces, that is, a leftward direction in the case of FIG. 11.

In the case of the situation as illustrated in FIG. 11, there is the monitor 16-1. In a case where the determination process of step S182 is performed, it is determined that the monitor 16 exists in a direction different from the direction in which the operator faces, and the processing proceeds to step S184. In step S184, a determination result indicating that there is a monitor that is unused is output.

In contrast, in a case where it is determined in step S182 that there is no monitor 16 in a direction different from the direction in which the operator faces, the processing proceeds to step S183, and a determination result indicating that there is no unused device is output.

In this manner, the use state of a device in the operation room is determined by using a video from the operating place camera 10.

Here, a case where the use state of the monitor 16 is determined has been described as an example; however, the use state of a device other than the monitor 16 may be determined in a similar manner.

Furthermore, the determination process can be executed in combination with one of processes of the flowcharts illustrated in FIGS. 6 to 9. For example, after the device arranged on the back side of the operator is specified by using a video from the operating place camera 10 illustrated in FIG. 10, the process of determining the use state of the device that has been specified may be executed. For example, after it is determined that the endoscope system 12 is located on the back side of the operator, the process of determining the use state of the endoscope system 12 illustrated in FIG. 6 or 7 may be executed.

The above-described processes can be executed in combination, and the use state can be more appropriately determined by executing the processes in combination.

The processes of determining the use states of the devices described as examples here are examples, and are not a description indicating limitation. For example, it is also possible to prepare a determination table for a device not illustrated here and determine the use state of the device.

Furthermore, here, a case where the process is performed with reference to the determination table (lookup table) has been described as an example; however, a determination may be made by using a learning method such as machine learning, for example, deep learning. For example, as described above, the use state is determined by analyzing video data; however, the use state described above may be determined by analyzing the video data, learning whether it is possible to determine that a device is being used or unused in what type of video, and using learning data obtained by the learning.

Learning data may be prepared for each device, learning data associated with the device may be selected on the basis of connected device information, and the use state of the device may be determined on the basis of the learning data that has been selected.

<Processing with Reference to Power Supply Management Table>

The description returns to the description with reference to the flowchart illustrated in FIG. 5. In step S74, a determination result as to whether the device is being used or unused is output by executing the process based on the determination table. The process in step S75 is executed by using the determination result.

In step S75, it is determined whether or not a determination result indicating that the device being used has been output. In step S75, in a case where the determination result indicates being used, the process in step S76 is skipped, and the process of the flowchart illustrated in FIG. 5 is terminated.

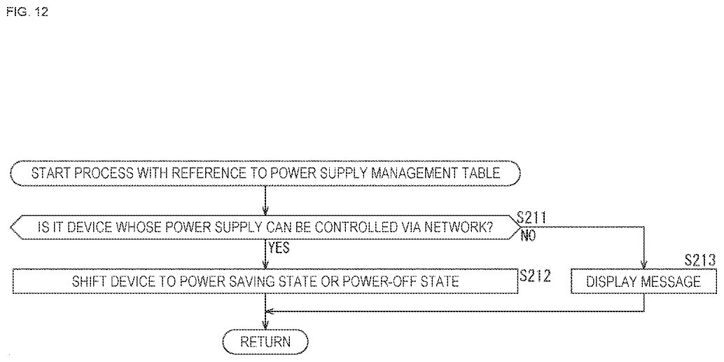

In contrast, in a case where it is determined in step S75 that it is determined that the determination result indicates that the device is unused, the processing proceeds to step S76. In step S76, the power supply control unit 84 (FIG. 3) executes the process with reference to the power supply management table. The process with reference to the power supply management table executed in step S76 will be described with reference to the flowchart illustrated in FIG. 12.

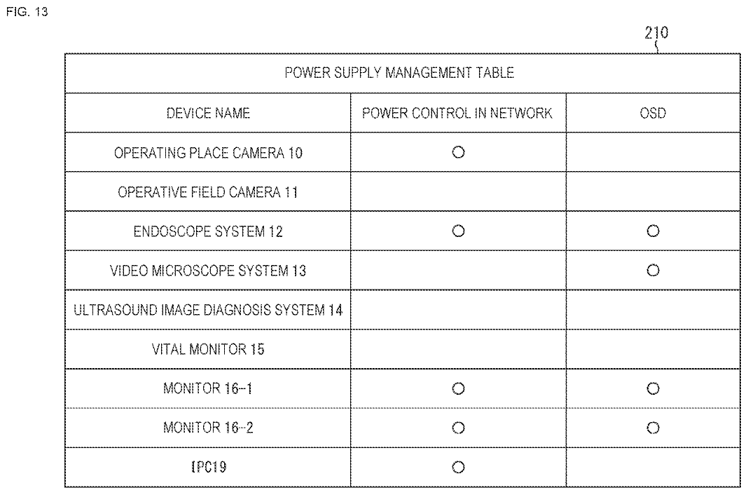

The power supply management table is, for example, a table in which a device and information indicating whether or not power control via a network can be performed are associated with each other as illustrated in FIG. 13. A power supply management table 210 illustrated in FIG. 13 illustrates an example of a table related to devices installed in the operation room A (FIG. 1). The power supply management table 210 as illustrated in FIG. 13 is prepared for each operation room.

In the power supply management table 210, items “device name”, “power control through network”, and “OSD” are provided. In the column “device name”, the name of a device installed in the operation room A is entered.

In the column “power control through network”, information on whether or not control such as power on/off and transition to the power saving state can be performed via the network is entered. In FIG. 13, a description will be given on the assumption that a circle is entered in a field corresponding to a device power control for which can be performed through the network.

“OSD” is an abbreviation of on-screen display, and information indicating whether or not the device has a function of displaying a predetermined message on a display is entered in the item “OSD”. In FIG. 13, a description will be given on the assumption that a circle is entered in a field corresponding to a device having the OSD function.

Note that, here, a device having the OSD function is a device that can receive a predetermined message related to power supply control as described later generated by the server 31 and display the predetermined message on the display. Alternatively, the device is a device that stores a plurality of predetermined messages and can read and display a message according to an instruction in a case where the instruction is given from the server 31.

Referring to FIG. 13, in the column “device name”, device names such as “operating place camera 10”, “operative field camera 11”, “endoscope system 12”, “video microscope system 13”, “ultrasound image diagnosis system 14”, “vital monitor 15”, “monitor 16-1”, “monitor 16-2”, and “IPC 19” are entered.

Among these devices, circles are entered in the fields “power control through network” associated with “operating place camera 10”, “endoscope system 12”, “monitor 16-1”, “monitor 16-2”, and “IPC 19”, which indicates that “operating place camera 10”, “endoscope system 12”, “monitor 16-1”, “monitor 16-2”, and “IPC 19” are devices for which power control can be performed through the network.

Furthermore, circles are entered in the fields of “OSD” associated with “endoscope system 12”, “video microscope system 13”, “monitor 16-1”, and “monitor 16-2”, which indicates that “endoscope system 12”, “video microscope system 13”, “monitor 16-1”, and “monitor 16-2” are devices having the OSD function.

The power supply management table 210 as described above is referred to in step S211 (FIG. 12), and it is determined whether or not the device is a device whose power supply can be controlled via the network. For example, in a case where the operating place camera 10 is set to be a processing target, it is determined in step S211 that the operating place camera 10 is a device whose power supply can be controlled via the network. Furthermore, for example, in a case where the video microscope system 13 is set to be a processing target, it is determined in step S211 that the device is not a device whose power supply can be controlled via the network.

In a case where it is determined in step S211 that the device is a device whose power supply can be controlled via the network, the processing proceeds to step S212. In step S212, since the device which is a processing target is a device whose power supply can be controlled via the network, an instruction to shift to the power saving state or the power-off state is output via the network to the device which is a processing target.

Whether the device is set to the power saving state or the power-off state may vary depending on the device, or the device may shift to either the power saving state or the power-off state regardless of the device.

Different instructions may be issued depending on the device. For example, in a case where a device which is a processing target is the operating place camera 10, an instruction to set the device to the power-off state may be issued to the operating place camera 10, and in a case where a device which is a processing target is the endoscope system 12, an instruction to set the device to the power saving state may be issued to the endoscope system 12.

Furthermore, for example, an instruction to set the device to the power saving state may be issued regardless of the device.

In contrast, in a case where it is determined in step S211 that the device is not a device whose power supply can be controlled via the network, the processing proceeds to step S213. In step S213, an instruction to display a message prompting the power saving state or power-off of the device which is a processing target is issued to the device having the OSD function.

For example, in a case where the device which is a processing target is the video microscope system 13, an instruction to display a message such as “Turn off if not in use” on the display included in the video microscope system 13 is issued. Alternatively, in a case where the device which is a processing target is the video microscope system 13, an instruction to display a message such as “Turn off the video microscope system if not in use” on the monitor 16 is issued.

Furthermore, for example, in a case where the device which is a processing target is the operative field camera 11, an instruction to display a message such as “Please turn off the operative field camera in a case where the operative field camera is not being used” on the monitor 16 is issued.

As described above, in a case where the device determined to be unused has the OSD function, an instruction to display a message is issued to the device. Alternatively, in a case where the device determined to be unused does not have the OSD function, an instruction to display a message for instructing the user to control the power supply of the device determined to be unused is issued to a device having the OSD function other than the device determined to be unused.

Note that an instruction from the server 31 is issued to the IPC 19 connected to a device to which it is desired to issue the instruction, and the instruction from the server 31 is transmitted via the IPC 19.

In the description with reference to the flowchart illustrated in FIG. 12, in step S212, in a case where the device determined to be unused is a device whose power supply can be controlled via the network, an instruction to shift the device to the power saving state or the power-off state is issued. However, a process similar to that in step S213 may be performed, that is, an instruction to display a message may be issued.

For example, a message such as “Turn off if not in use” may be displayed on the device or the monitor 16 determined to be unused, and the user who has noticed the message may be allowed to manage the power supply thereof.

Furthermore, in a case where a predetermined time has elapsed after such a message is displayed and it is continuously determined that the device is unused, the power supply may be controlled via the network and the device may be shifted to the power-off state. In this case, for example, a message that allows the user to recognize that the power will be turned off and the time until the power will be turned off, such as “Power is turned off in five minutes”, may be displayed.

By executing the above-described process, the IPC 19 connected to the device to which an instruction to turn off the device or shift the device to the power saving state has been issued may also receive the same instruction, and may be turned off or shifted to the power saving state. For example, in a case where an instruction to shift to the power saving state is issued to the endoscope system 12, an instruction to shift to the power saving state may also be issued to the IPC 19-1 connected to the endoscope system 12.

That is, the power supply control of the IPC 19 can also be performed by the above-described process.

Furthermore, as power supply control of the IPC 19 itself, power consumption of the IPC 19 may be reduced by decreasing the frame rate or decreasing the image quality. Such power supply control of the IPC 19 itself may be performed, for example, when the capacity of the UPS 51 decreases.

A means that can prevent the device from shifting to the power saving state or the power-off state on the user side may be provided. For example, the user checks that the device shifts to the power saving state or the power-off state from the message displayed; however, the user may not desire to shift the device to the power saving state or the power-off state. In such a case, for example, a means capable of avoiding the device from shifting to the power saving state or the power-off state through an operation or the like of a user interface (UI) performed by a user by using voice, a hand gesture, or a touch panel may be provided.

Alternatively, the device may be prevented from shifting to the power saving state or the power-off state unless there is, for example, an operation or the like of the user interface (UI) performed by the user by using a voice, a hand gesture, or the touch panel. That is, by displaying a message or the like, it is recommended that the user set the power saving state or the power-off state; however, permission of the user may be required to finally shift to the power saving state or the power-off state.

In this manner, if the process with reference to the power supply management table 210 is executed in step S76 (FIG. 5), the processing of the server 31 is terminated.

As described, by determining the use state of the device and controlling the power supply of the device determined to be unused, for example, even in a case where the following situation occurs, control for appropriately suppressing power consumption can be performed.

For example, in a case where a medical operation using the endoscope system 12 has been performed but has shifted to laparotomy, it is assumed that a situation in which a state where the endoscope system 12 is turned on is maintained even though the endoscope system 12 is not being used has occurred.

In a case where such a situation occurs, according to the present technology described above, it is possible to detect that the endoscope system 12 is unused and turn off the endoscope system 12.

### <Second Process of Server 31>

The process of the server 31 described with reference to the flowchart illustrated in FIG. 5 is the process of determining the use state of the device which is a processing target, and basically, a case where the determination is made for each individual device has been described as an example. As the second process of the server 31, a case where power of a predetermined device is controlled on the basis of information from a plurality of devices will be described as an example.

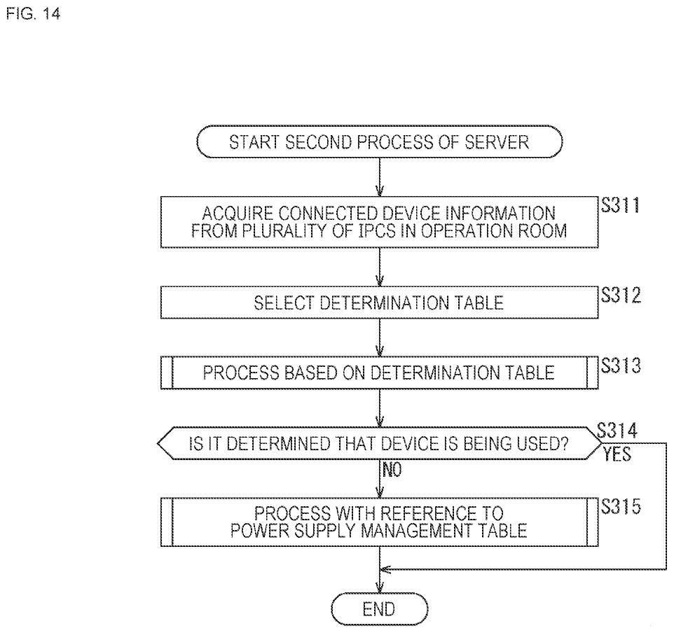

FIG. 14 is a flowchart for explaining the second process of the server 31.

In step S311, the server 31 acquires connected device information from a plurality of devices (IPCs 19) installed in the operation room A, for example. In step S312, a determination table is selected. The determination table selected in step S312 is a table for detecting an unused device (device set to the power saving state) by using information from the plurality of devices.

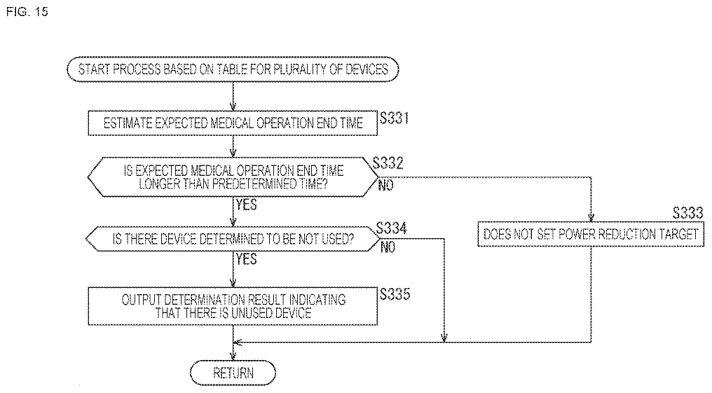

In step S313, a process based on the selected determination table is executed. The process based on the determination table executed in step S313 will be described with reference to the flowchart of FIG. 15.

In step S331, an expected medical operation end time is estimated. For example, in a case where a time required for a medical operation is scheduled in advance, the expected end time can be estimated with reference to the scheduling information.

Furthermore, for example, a video from the operative field camera 11 and a video from the endoscope system 12 may be analyzed, a progress status of the medical operation may be determined, and the expected end time may be estimated. Furthermore, the expected end time may be set by a report from the operator.

When the expected medical operation end time is estimated in step S331, the estimation result is used to determine in step S332 whether or not the expected medical operation end time is longer than a predetermined time.

In a case where it is determined in step S332 that the expected medical operation end time is shorter than the predetermined time, the processing proceeds to step S333. In step S333, a determination result indicating that there is no device to be subjected to power amount reduction is output.

In contrast, in a case where it is determined in step S332 that the expected medical operation end time is longer than the predetermined time, the processing proceeds to step S334. In step S334, it is determined whether or not there is a device determined to be not used.

In a case where the expected medical operation end time cannot be estimated (in a case where the expected end time point is undetermined), it is determined in step S332 that the expected medical operation end time is longer than the predetermined time.

In the processes of steps S331 to S334, in a case where the remaining medical operation time is shorter than the predetermined time, the medical operation is continued in the state at that time without setting a power reduction target, and in a case where the remaining operation time is longer than the predetermined time, a device that is not being used is detected in order to reduce power. In this manner, it is determined whether or not a power reduction target is determined according to the operation time.

Here, a case where it is determined whether or not to set a power reduction target according to the expected medical operation end time will be described as an example; however, the process may be performed by setting a power reduction target regardless of the medical operation time. That is, the process in which the processes of steps S331 to S333 is omitted can also be executed.

In step S334, it is determined whether or not there is a device determined to be not used. This determination can be performed, for example, on the basis of the flowcharts (determination tables corresponding to predetermined devices) illustrated in FIGS. 6 to 10.

In a case where it is determined in step S334 that there is no device determined to be not used, the processing proceeds to step S314 (FIG. 14). In contrast, in a case where it is determined in step S334 that there is a device determined to be not used, the processing proceeds to step S335.

In step S335, a determination result indicating that there is an unused device is output, and the processing proceeds to step S314 (FIG. 14).

In step S314, it is determined whether or not the device is being used. In a case where the processing has reached step S314 (FIG. 14) from step S333 (FIG. 15), or in a case where it is determined in step S334 (FIG. 15) that there is no device determined to be not used and the processing has reached step S314 (FIG. 14), it is determined in step S314 that the devices are being used.

In contrast, in a case where it is determined in step S334 (FIG. 15) that there is a device determined to be not used and the processing has reached step S314 (FIG. 14), NO is determined in step S314 and the processing proceeds to step S315.

In step S315, the process with reference to the power supply management table is executed. The process in step S315 is performed similarly to step S76 (FIG. 5), and can be performed on the basis of the flowchart illustrated in FIG. 12, and thus, the description thereof is omitted here. In step S315, the process with reference to the power supply management table is performed on the device determined to be unused in step S334 (FIG. 15).

By executing such a process, as an example, the following settings are configured in the respective operation rooms A to C.

Operation room A: Since the expected medical operation end time is about three hours and the devices are being used, supply of power is maintained.

Operation room B: Since the expected medical operation end time is about five hours, and there is a monitor 16 that can be determined to be not used on the back side of the operator, the monitor 16 is turned off.

Operation room C: The expected medical operation end time is undetermined, and the endoscope system 12 is turned on. However, since it is determined that the endoscope system 12 images an area other than the inside of a body cavity, the endoscope system 12 shifts to the power saving state.

As described, according to the present technology, a device that is not being used can be detected. This detection can be performed by using a video captured by the device. Furthermore, it is possible to control supply of power to the device not determined to be used.

By controlling supply of power to the device determined to be not used, power to be supplied can be reduced. For example, if supply of power is controlled by applying the present technology when power is supplied by the UPS 51 having limited capacity, it is possible to appropriately detect a device that does not require power while supplying power to a device that requires power, and to reduce supply of power. Therefore, even in an emergency, it is possible to maintain a state in which power can be supplied to a device such as a medical device and the device can be used in a longer and appropriate state.

### <Function of Medical Power Supply System, Second Operation>

Another operation of the medical power supply system illustrated in FIG. 1 will be described. There is a possibility that operation of the medical power supply system described with reference to FIG. 5 increases the processing load on the server 31.

According to the operation of the medical power supply system described with reference to FIG. 5, the server 31 needs to receive and process video data from a plurality of devices installed in a plurality of operation rooms. As a result, if the number of devices increases, the processing load of the server 31 increases accordingly. Therefore, functions and operation of the medical power supply system that performs some of the above-described processes on the IPC 19 side and reduces the processing load of the server 31 will be described.

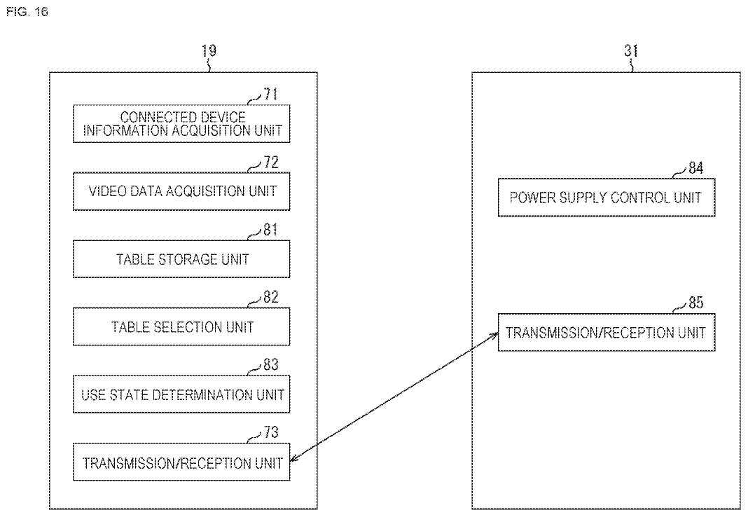

FIG. 16 is a diagram illustrating a functional configuration example of the medical power supply system that performs second operation. An IPC 19 includes a connected device information acquisition unit 71, a video data acquisition unit 72, a table storage unit 81, a table selection unit 82, a use state determination unit 83, and a transmission/reception unit 73. A server 31 includes a power supply control unit 84 and a transmission/reception unit 85.

If the functional configuration example of the medical power supply system that performs the second operation illustrated in FIG. 16 is compared with the functional configuration example of the medical power supply system that performs the first operation illustrated in FIG. 3, the former differs from the latter in that the IPC 19 includes the functions of the table storage unit 81, the table selection unit 82, and the use state determination unit 83 included in the server 31 in the former.

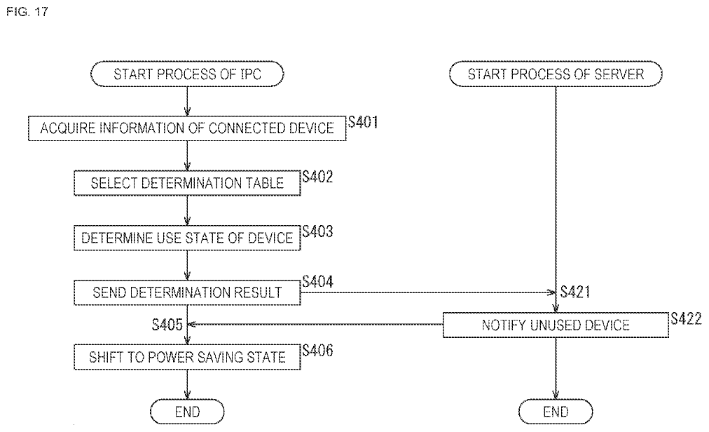

FIG. 17 is a diagram for explaining second operation of the medical power supply system.

In step S401, the IPC acquires information on the device to which the IPC 19 is connected. The process in step S401 can be performed similarly to the process executed by the IPC 19 in step S11 (FIG. 4), and thus the description thereof will be omitted.

In step S402, the IPC selects a determination table corresponding to the device to which the IPC 19 is connected. The process in step S402 can be performed similarly to the process executed by the server 31 in step S32 (FIG. 4). The IPC 19 performs a process of selecting a determination table, which is performed by the server 31 in the above description.

The processes in steps S401 and S402 may be performed once at a predetermined timing, and then the determination table that has been selected may be used. For example, the processes of steps S401 and S402 may be executed when a device is connected to the IPC 19, and once the determination table is set, the processing may be executed from step S403. Furthermore, the processes of steps S401 and S402 may be executed, for example, each time a device is turned on.

In step S403, the use state of the device is determined on the basis of the determination table that has been selected. The process in step S403 can be performed similarly to the process executed by the server 31 in step S34 (FIG. 4).

As described with reference to FIG. 2, the IPC 19 has a function of acquiring video data from the connected device, packetizing the video data into an IP packet, and transmitting the IP packet to the server 31. Since the IPC 19 has a function of acquiring video data, the IPC 19 can perform processing up to analyzing the video data that has been acquired and determining whether or not the connected device is being used.

The process of determining the use state of the device executed in step S403 can be performed by applying any of the processes based on the determination tables of the predetermined devices described with reference to FIGS. 6 to 10.

For example, if the IPC 19-1 is described as an example, the IPC 19-1 is connected to the endoscope system 12 (FIG. 1). Therefore, the IPC 19-1 acquires information of the endoscope system 12 as connected device information. Then, the IPC 19-1 selects the determination table for the endoscope system 12.

The process performed when the determination table for the endoscope system 12 is selected is the process of the flowchart illustrated in FIG. 6 or the process of the flowchart illustrated in FIG. 7. The IPC 19-1 determines whether or not the endoscope system 12 is being used by executing the process of the flowchart illustrated in FIG. 6 or/and the process of the flowchart illustrated in FIG. 7.

Since an IPC 19-2 is connected to the video microscope system 13 (FIG. 1), the IPC 19-2 selects the determination table for the video microscope system 13, and performs the process based on the determination table. That is, the IPC 19-2 determines whether or not the video microscope system 13 is being used by executing the process of the flowchart illustrated in FIG. 6 or/and the process of the flowchart illustrated in FIG. 7.

Since an IPC 19-3 is connected to the ultrasound image diagnosis system 14 (FIG. 1), the IPC 19-3 selects a determination table for the ultrasound image diagnostic system 14, and the process based on the determination table is performed. That is, the IPC 19-3 determines whether or not the ultrasound image diagnosis system 14 is being used by executing the process of the flowchart illustrated in FIG. 8.

Since an IPC 19-4 is connected to the vital monitor 15 (FIG. 1), a determination table for the vital monitor 15 is selected and the process based on the determination table is performed. Although the determination table for the vital monitor 15 is not described, for example, it is determined whether or not the vital monitor 15 is being used by determining whether or not a vital signal is obtained.

Although a case where no IPC 19 is connected to the operating place camera 10 and the operative field camera 11 has been described as an example in the system configuration example illustrated in FIG. 1, a configuration may be possible in which the IPCs 19 are connected to the operating place camera 10 and the operative field camera 11, respectively.

Furthermore, in a case where the IPC 19 is connected to the operating place camera 10, the IPC 19 connected to the operating place camera 10 selects a determination table for the operating place camera 10 and performs a process based on the determination table. That is, the IPC 19 connected to the operating place camera 10 detects a device not being used in the operation room by executing the process of the flowchart illustrated in FIG. 10.

Furthermore, in a case where the IPC 19 is connected to the operative field camera 11, the IPC 19 connected to the operative field camera 11 selects a determination table for the operative field camera 11 and performs a process based on the determination table. That is, the IPC 19 connected to the operative field camera 11 determines whether or not the operative field camera 11 is being used by executing the process of the flowchart illustrated in FIG. 9.

Alternatively, as illustrated in FIG. 1, a configuration may be possible in which no IPC 19 is connected to the operating place camera 10 and the operative field camera 11, and processing for such a device to which no IPC 19 is connected may be performed by the server 31. That is, the processing on the device to which no IPC 19 is connected may be performed on the server 31 side by applying the first process of the server illustrated in FIG. 5 or/and the second process of the server illustrated in FIG. 14.

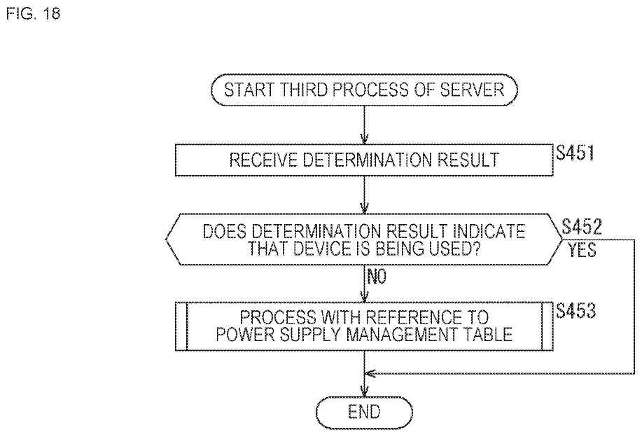

As the processing of the server 31, processing can be executed by combining the first process illustrated in FIG. 5, the second process of the server illustrated in FIG. 14, and a third process of the server described with reference to FIG. 18.

Returning to the description with reference to FIG. 17, the determination result regarding the use state of the device determined in step S403 is transmitted to the server 31 in step S404. The determination result to be transmitted is information indicating that the device is being used or unused, and may be, for example, information of 0 or 1 such as a flag.

As described, the processing up to determination of the use state of a device performed by the server 31 is performed on the IPC 19 side. Processing up to determination of the use state of a device is performed on the IPC 19 side, and therefore the processing load on the server 31 can be reduced.

In step S421, the server 31 receives the determination result transmitted from the IPC 19. In a case where the server 31 receives a determination result and the determination result indicates that the device is unused, the server 31 notifies the unused device in step S422. The processing in the server 31 will be described later with reference to FIG. 18.

In step S422, the IPC 19 that has received the notification to the unused device transmitted from the server 31 in step S405 advances the processing to step S406. In step S406, the IPC 19 shifts the connected device to the power saving state. Note that, here, an example of shifting to the power saving state has been described. However, in a case where the instruction from the server 31 is a notification including, for example, an instruction to display a message or to turn off power, processing based on the instruction is executed in step S406.

### <Third Process of Server 31>

The third process performed by the server 31 will be described with reference to FIG. 18.

In step S451, the server 31 receives the determination result from the IPC 19. The determination result is information indicating whether the device is being used or unused. In step S452, it is determined whether or not the determination result is information indicating that the device is being used.

In a case where it is determined in step S452 that the determination result indicates that the device is being used, the processing in the server 31 for the device which has sent the determination result is terminated. In contrast, in a case where it is determined in step S452 that the determination result indicates that the device is unused, the processing proceeds to step S453.

In step S453, a process with reference to the power supply management table is executed. The process with reference to the power supply management table executed in step S453 can be performed on the basis of the flowchart illustrated in FIG. 12, and the description thereof will be omitted here so as not to be repeated.

As described, in the server 31, since power supply control is performed for the device that has issued the determination result indicating that the device is unused, the IPC 19 may transmit the determination result to the server 31 only when the determination indicating that the device is unused is issued.

### <Fourth Process of Server 31>

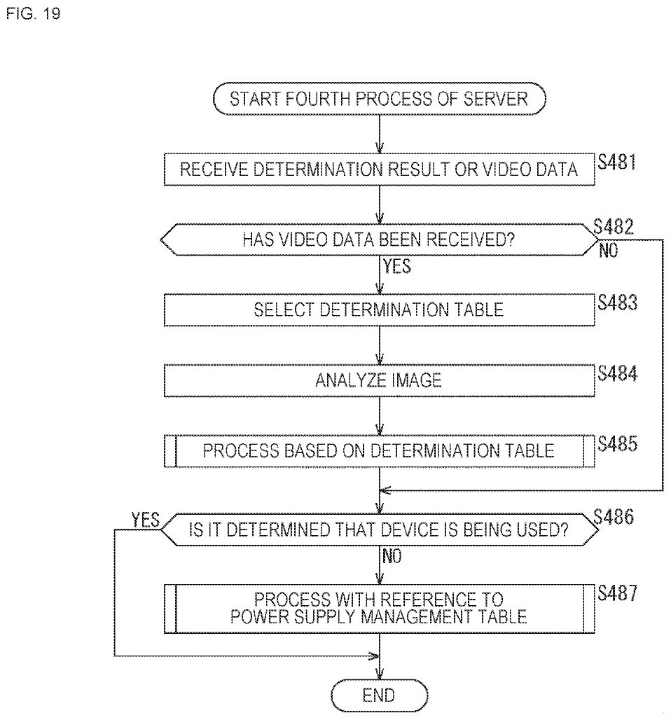

A fourth process performed by the server 31 will be described with reference to FIG. 19. The fourth process is a process in a case where the first process illustrated in FIG. 5, the second process of the server illustrated in FIG. 14, and the third process of the server illustrated in FIG. 18 are combined.

In step S481, the server 31 receives a determination result or video data. In step S482, it is determined whether or not video data is received.

In a case where it is determined in step S482 that video data is received, processes in and after step S483 are performed. The process when video data is received can be performed by performing the first process of the server illustrated in FIG. 5 or the second process of the server illustrated in FIG. 14.

Since the processes in steps S483 to S487 can be performed similarly to the processes in steps S72 to S76 (FIG. 5), the description thereof will be omitted.

In contrast, in a case where it is determined in step S482 that what is received is not video data, in other words, in a case where it is determined that a determination result is received, the processes in and after step S486 are performed. The processes performed when the determination result is received can be performed by performing the third process illustrated in FIG. 18.

Since the processes of steps S486 and S487 can be performed similarly to the processes of steps S452 and S453 (FIG. 18), the description thereof will be omitted.

Although not illustrated, in a case where it is determined in step S482 that what is received is a determination result, the processing may proceed to step S485. Then, in step S485, the processing may be performed on the basis of a table for making a determination by using information from a plurality of devices in an integrated manner. For example, the process based on the table for a plurality of devices illustrated in FIG. 15 can be executed.

As described, according to the present technology, a device that is not being used can be detected. This detection can be performed by using a video captured by the device. This detection can be performed by the IPC 19 connected to the device. Then, processing of the server 31 enables supply of power to the device determined to be not used to be controlled. Furthermore, the processing load on the server 31 can be reduced.

By controlling supply of power to the device determined to be not used, power to be supplied can be reduced. For example, if supply of power is controlled by applying the present technology when power is supplied by the UPS 51 having limited capacity, it is possible to appropriately detect a device that does not require power while supplying power to a device that requires power, and to reduce supply of power. Therefore, even in an emergency, it is possible to maintain a state in which power can be supplied to a device such as a medical device and the device can be used in a longer and appropriate state.

### <Function of Medical Power Supply System, Third Operation>

Another operation of the medical power supply system illustrated in FIG. 1 will be described. Although the first operation and the second operation of the medical power supply system described above include processing on the server 31 side, the processing may be performed only on the IPC 19 side.

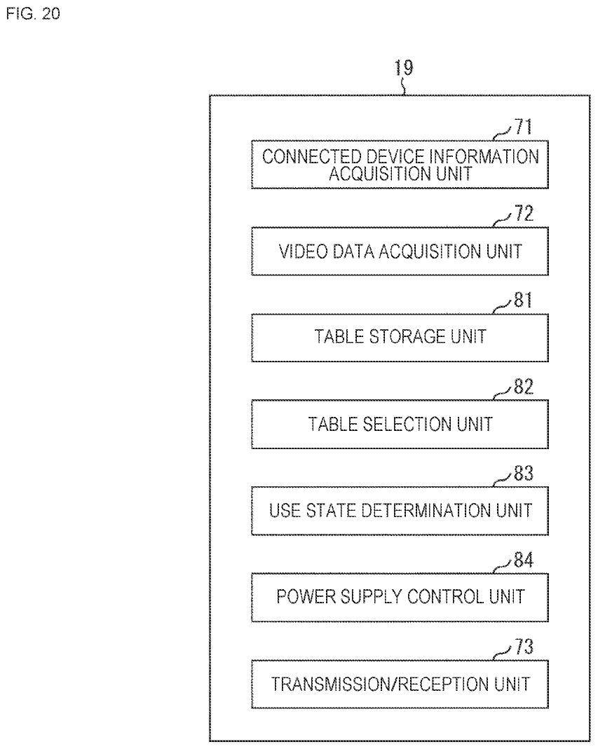

FIG. 20 is a diagram illustrating a functional configuration example of the medical power supply system that performs third operation. In the medical power supply system that performs the third operation, since the processing is performed on the IPC 19 side, FIG. 20 illustrates a functional configuration example of the IPC 19. The IPC 19 includes a connected device information acquisition unit 71, a video data acquisition unit 72, a table storage unit 81, a table selection unit 82, a use state determination unit 83, a power supply control unit 84, and a transmission/reception unit 73.

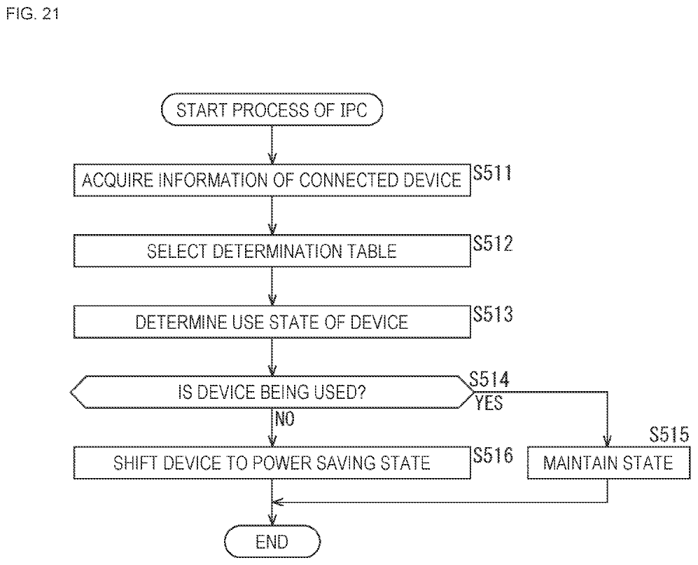

FIG. 21 is a diagram for explaining the third operation of the medical power supply system; however, is a diagram for explaining the processing in the IPC 19 since the processing is performed only on the IPC 19 side.

In step S511, the IPC 19 acquires information on the device to which the IPC 19 is connected. In step S512, the IPC 19 selects a determination table corresponding to the device to which the IPC 19 is connected. In step S513, the use state of the device is determined on the basis of the determination table that has been selected.

Since the processes in steps S511 to S513 can be performed similarly to the processes of steps S401 to S403 (FIG. 17), the description thereof is omitted here.

In step S514, it is determined whether or not the connected device is being used. In a case where it is determined in step S514 that the connected device is being used, the processing proceeds to step S515. In step S515, the state at that time is maintained. That is, the processing at that time is continuously performed without performing control regarding the power supply.

In contrast, in a case where it is determined in step S514 that the connected device is not being used, in other words, in a case where it is determined that the connected device is unused, the processing proceeds to step S516. In step S516, the IPC 19 shifts the connected device to the power saving state. In a case where the device is subjected to power supply control via a network, the device is shifted to a power saving state or is shifted to a power-off state. Furthermore, a message may be displayed on a display.

The message may be displayed on a display of a connected device, or may be displayed on another device such as the monitor 16. In a case where the message is displayed on another device, the IPC 19 connected to the device is instructed to display the message. An instruction may be given or received by IPCs 19, or an instruction may be given or received via the server 31.

A configuration is possible in which in a case where the connected device is turned off or the connected device is shifted to the power saving state, the IPC 19 itself is also turned off or shifted to the power saving state.

In this manner, it is also possible to adopt a configuration in which the use state of the connected device is determined and processing up to power supply control according to the use state is performed on the IPC 19 side.

As described, according to the present technology, the use state of the connected device can be detected in the IPC 19. This detection can be performed by using a video captured by the device. In addition, control regarding power consumption can be performed depending on the use state. Furthermore, the processing load on the server 31 can be reduced. In addition, the amount of data transmitted and received via the network can be reduced more than, for example, that in the first operation of the medical power supply system.

By controlling supply of power to the device determined to be not used, power to be supplied can be reduced. For example, if supply of power is controlled by applying the present technology when power is supplied by the UPS 51 having limited capacity, it is possible to appropriately detect a device that does not require power while supplying power to a device that requires power, and to reduce supply of power. Therefore, even in an emergency, it is possible to maintain a state in which power can be supplied to a device such as a medical device and the device can be used in a longer and appropriate state.

### <Example of Execution by Software>

Incidentally, the series of processes described above can be executed by hardware, but can also be executed by software. In a case where the series of processes is executed by software, a program that constitutes the software is installed from a recording medium to a computer built into dedicated hardware or, for example, a general-purpose computer or the like to which various programs are installed so as to be able to execute various functions.

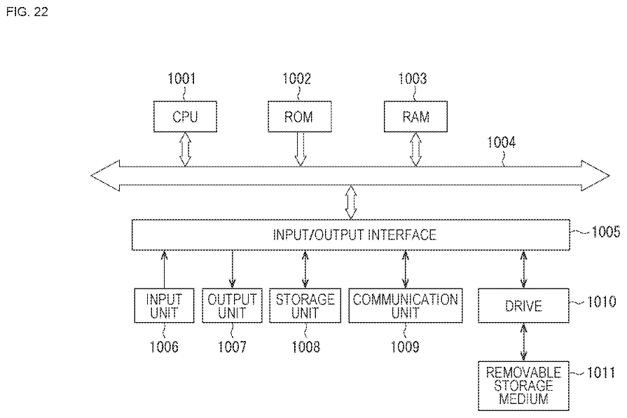

FIG. 22 illustrates a configuration example of a general-purpose computer. This personal computer includes a central processing unit (CPU) 1001. An input/output interface 1005 is connected to the CPU 1001 via a bus 1004. A read only memory (ROM) 1002 and a random access memory (RAM) 1003 are connected to the bus 1004.

An input unit 1006 including an input device such as a keyboard or a mouse by which a user inputs an operation command, an output unit 1007 that outputs a processing operation screen or an image of a processing result to a display device, a storage unit 1008 including a hard disk drive or the like that stores a program and various data, and a communication unit 1009 that includes a local area network (LAN) adapter or the like and that executes a communication process via a network a typical example of which is the Internet are connected to the input/output interface 1005. Furthermore, a drive 1010 that reads and writes data from and to a removable storage medium 1011 such as a magnetic disc (including a flexible disc), an optical disc (including a compact disc-read only memory (CD-ROM), a digital versatile disc (DVD)), a magneto-optical disc (including a Mini Disc (MD)), a semiconductor memory, or the like is connected to the input/output interface 1005.

The CPU 1001 executes various processes according to a program stored in the ROM 1002, or a program read from the removable storage medium 1011 such as a magnetic disc, an optical disc, a magneto-optical disc, a semiconductor memory, or the like, installed in the storage unit 1008, and loaded from the storage unit 1008 into the RAM 1003. The RAM 1003 also appropriately stores data or the like necessary for the CPU 1001 to execute various processes.

In the computer configured as described above, for example, the CPU 1001 loads the program stored in the storage unit 1008 into the RAM 1003 via the input/output interface 1005 and the bus 1004 and executes the program, and thus the above-described series of processes is performed.

The program executed by the computer (CPU 1001) can be provided by being recorded on, for example, the removable storage medium 1011 as a package medium or the like. Furthermore, the program can be provided via a wired or wireless transmission medium such as a local area network, the Internet, or digital satellite broadcasting.

In the computer, the program can be installed into the storage unit 1008 via the input/output interface 1005 by inserting the removable storage medium 1011 into the drive 1010. Furthermore, the program can be received by the communication unit 1009 via a wired or wireless transmission medium and can be installed in the storage unit 1008. In addition, the program can be installed in advance into the ROM 1002 or the storage unit 1008.

Note that the program executed by the computer may be a program that performs processes in chronological order according to the order described in the present Description, or may be a program that performs processes in parallel, or at necessary timing, such as when a call is made.

Furthermore, in the present Description, a system represents an entire apparatus including a plurality of apparatuses.

Note that the effects described in the present Description are illustrations only and not limited, and may have other effects.

Note that the embodiments of the present technology are not limited to the above-described embodiments, and various modifications can be made without departing from the scope of the present technology.

Note that the present technology can also be configured as follows.

(1)

A medical power supply system including: a first information processing apparatus that is connected to a medical device installed in an operation room; and a second processing apparatus that exchanges data with the first information processing apparatus, the medical power supply system further including:

an acquisition unit that acquires data from the medical device;

a determination unit that determines a use state of the medical device on the basis of the data; and

a control unit that controls a power supply state of at least one medical device in the operation room on the basis of the use state of the medical device determined by the determination unit.

(2)

The medical power supply system according to (1),

in which data acquired from the medical device is video data,

the determination unit determines the use state of the medical device by analyzing the video data, and

the control unit controls a power supply of a medical device determined to be unused in a determination result obtained by the determination unit.

(3)

The medical power supply system according to (1) or (2),

in which the power supply state of the at least one medical device is controlled by the control unit when power is supplied to the medical device installed in the operation room by an uninterruptible power supply.

(4)

An information processing apparatus including:

an acquisition unit that acquires data from a medical device;

a determination unit that analyzes the data and determines a use state of the medical device; and

a control unit that controls a power supply state of the medical device on the basis of the use state of the medical device determined by the determination unit.

(5)

The information processing apparatus according to (4),

in which the data is video data,

the determination unit determines a use state of the medical device by analyzing the video data, and

the control unit controls a power supply of a medical device determined to be unused in a determination result obtained by the determination unit.

(6)

The information processing apparatus according to (5),

in which the determination unit determines a use state of the medical device by determining whether or not a video is captured while the medical device is being used.

(7)

The information processing apparatus according to (4),

in which the data is data regarding a use state of the medical device,

the determination unit determines a use state of the medical device on the basis of the data, and

the control unit controls a power supply of a medical device determined to be unused in a determination result obtained by the determination unit.

(8)

The information processing apparatus according to any one of (4) to (7),

in which the control unit issues an instruction to shift a medical device determined to be unused to a power saving state or a power-off state, or an instruction to display a message for prompting turning off of a medical device determined to be unused.

(9)

The information processing apparatus according to (8),

in which the message is displayed on a monitor other than the medical device, and the instruction is issued to the monitor.

(10)

The information processing apparatus according to any one of (4) to (9),

in which the determination unit performs the determining with reference to a determination table for the medical device.

(11)

The information processing apparatus according to any one of (4) to (10),

in which the determination unit detects an unused medical device in an operation room on the basis of data from a plurality of the medical devices.

(12)

The information processing apparatus according to any one of (4) to (11),

in which the determination unit analyzes video data from the medical device and detects a monitor in a direction different from a line-of-sight direction of an operator, and

the control unit controls a power supply of the monitor that has been detected.

(13)

An information processing method performed by an information processing apparatus that controls a power supply state of a medical device, the information processing method including:

acquiring data from the medical device;

analyzing the data and determining a use state of the medical device; and

controlling a power supply state of the medical device on the basis of the use state of the medical device that has been determined.

(14)

An image processing apparatus including:

an acquisition unit that acquires video data captured by a medical device;

a determination unit that analyzes the video data and determines a use state of the medical device; and

a control unit that controls a power supply state of the medical device on the basis of the use state of the medical device determined by the determination unit.

(15)

The image processing apparatus according to (14),

in which the determination unit determines a use state of the medical device by determining whether or not a video is captured while the medical device is being used.

(16)

The image processing apparatus according to (14) or (15),

in which in a case where the determination unit determines that the medical device is unused, the control unit shifts the medical device that is connected to a power saving state or a power-off state.

(17)

The image processing apparatus according to any one of (14) to (16),

in which in a case where the determination unit determines that the medical device is unused, the control unit issues, to the medical device, an instruction to display a message prompting turning off of the medical device that is connected.

(18)

The image processing apparatus according to any one of (14) to (17),

in which the determination unit performs the determining with reference to a determination table for the medical device that is connected.

(19)

An image processing method performed by an image processing apparatus that is connected to a medical device and processes video data from the medical device, the image processing method including:

acquiring video data captured by the medical device;

analyzing the video data and determining a use state of the medical device; and

controlling a power supply state of the medical device on the basis of the use state of the medical device that has been determined.

### REFERENCE SIGNS LIST

10 Operating place camera
11 Operative field camera
12 Endoscope system
13 Video microscope system
14 Ultrasound image diagnosis system
15 Vital monitor
16 Monitor
17 IP switcher
18 Controller
31 Server
51 UPS
71 Connected device information acquisition unit
72 Video data acquisition unit
73 Transmission/reception unit
81 Table storage unit
82 Table selection unit
83 Use state determination unit
84 Power supply control unit
85 Transmission/reception unit
210 Power supply management table

1. A medical power supply system comprising: a first information processing apparatus that is connected to a medical device installed in an operation room; and a second processing apparatus that exchanges data with the first information processing apparatus, the medical power supply system further comprising:
an acquisition unit that acquires data from the medical device;
a determination unit that determines a use state of the medical device on a basis of the data; and
a control unit that controls a power supply state of at least one medical device in the operation room on a basis of the use state of the medical device determined by the determination unit.

2. The medical power supply system according to claim 1,
wherein data acquired from the medical device is video data,
the determination unit determines a use state of the medical device by analyzing the video data, and
the control unit controls a power supply of a medical device determined to be unused in a determination result obtained by the determination unit.

3. The medical power supply system according to claim 1,
wherein the power supply state of the at least one medical device is controlled by the control unit when power is supplied to the medical device installed in the operation room by an uninterruptible power supply.

4. An information processing apparatus comprising:
an acquisition unit that acquires data from a medical device;
a determination unit that analyzes the data and determines a use state of the medical device; and
a control unit that controls a power supply state of the medical device on a basis of the use state of the medical device determined by the determination unit.

5. The information processing apparatus according to claim 4,
wherein the data is video data,
the determination unit determines a use state of the medical device by analyzing the video data, and
the control unit controls a power supply of a medical device determined to be unused in a determination result obtained by the determination unit.

6. The information processing apparatus according to claim 5,
wherein the determination unit determines a use state of the medical device by determining whether or not a video is captured while the medical device is being used.

7. The information processing apparatus according to claim 4,
wherein the data is data regarding a use state of the medical device,
the determination unit determines a use state of the medical device on a basis of the data, and
the control unit controls a power supply of a medical device determined to be unused in a determination result obtained by the determination unit.

8. The information processing apparatus according to claim 4,
wherein the control unit issues an instruction to shift a medical device determined to be unused to a power saving state or a power-off state, or an instruction to display a message for prompting turning off of a medical device determined to be unused.

9. The information processing apparatus according to claim 8,
wherein the message is displayed on a monitor other than the medical device, and the instruction is issued to the monitor.

10. The information processing apparatus according to claim 4,
wherein the determination unit performs the determining with reference to a determination table for the medical device.

11. The information processing apparatus according to claim 4,
wherein the determination unit detects an unused medical device in an operation room on a basis of data from a plurality of the medical devices.

12. The information processing apparatus according to claim 4,
wherein the determination unit analyzes video data from the medical device and detects a monitor in a direction different from a line-of-sight direction of an operator, and
the control unit controls a power supply of the monitor that has been detected.

13. An information processing method performed by an information processing apparatus that controls a power supply state of a medical device, the information processing method comprising:
acquiring data from the medical device;
analyzing the data and determining a use state of the medical device; and
controlling a power supply state of the medical device on a basis of the use state of the medical device that has been determined.

14. An image processing apparatus comprising:
an acquisition unit that acquires video data captured by a medical device;
a determination unit that analyzes the video data and determines a use state of the medical device; and
a control unit that controls a power supply state of the medical device on a basis of the use state of the medical device determined by the determination unit.

15. The image processing apparatus according to claim 14,
wherein the determination unit determines a use state of the medical device by determining whether or not a video is captured while the medical device is being used.

16. The image processing apparatus according to claim 14,
wherein in a case where the determination unit determines that the medical device is unused, the control unit shifts the medical device that is connected to a power saving state or a power-off state.

17. The image processing apparatus according to claim 14,
wherein in a case where the determination unit determines that the medical device is unused, the control unit issues, to the medical device, an instruction to display a message prompting turning off of the medical device that is connected.

18. The image processing apparatus according to claim 14,
wherein the determination unit performs the determining with reference to a determination table for the medical device that is connected.

19. An image processing method performed by an image processing apparatus that is connected to a medical device and processes video data from the medical device, the image processing method comprising:
acquiring video data captured by the medical device;
analyzing the video data and determining a use state of the medical device; and
controlling a power supply state of the medical device on a basis of the use state of the medical device that has been determined.
